(19) United States
(12) Patent Application Publication
(10) Pub. No.: US 2021/0249128 A1
(43) Pub. Date: Aug. 12, 2021

(54) MODULAR APPLICATION DEVELOPMENT PLATFORM AND SYSTEM

(71) Applicant: Natalia Matveeva, Klaipeda (LT)

(72) Inventor: Natalia Matveeva, Klaipeda (LT)

(21) Appl. No.: 16/844,980

(22) Filed: Apr. 9, 2020

Related U.S. Application Data
(60) Provisional application No. 62/971,538, filed on Feb. 7, 2020.

(51) Int. Cl. G16H 40/63; G06N 5/02; G06F 8/30

(52) U.S. Cl. CPC G16H 40/63; G06F 8/30; G06N 5/02

(57) ABSTRACT

A platform configured to facilitate the creation of health-centric mobile-device and web-based applications is described. The platform employs pre-designed modules designed to expedite the application creation process, and to ensure accurate data logging, tracking, and analysis over time on a wide variety of health-based factors. Health-oriented companies wishing to craft an application for use by their users can easily create a customized application with wide-ranging tracking and analysis features, employing AI, without the need for knowledge of programing or conventional application development procedures.

### CONTINUITY

This application is a non-provisional patent application of provisional patent application No. 62/971,538, filed on Feb. 7, 2020, as well as of provisional patent application No. 62/834,204, filed on Apr. 9, 2019, and priority is claimed thereto.

### FIELD OF THE PRESENT INVENTION

The present invention relates to the field of modular application development, and more specifically relates to health-focused platforms configured to facilitate the development of customized mobile applications geared to collect information, track development/performance, and analyze health-based data and input to reflect and assist with the well-being of users.

### BACKGROUND OF THE PRESENT INVENTION

Today, it is known that individuals are becoming more informed about their health. Health awareness has become more popular than ever as individuals from all age groups are learning to live healthier, longer, and more active lives. Recently, personal technology has advanced to the point where individuals may easily track specific health metrics, ranging from weight and diet, to exercises performed in real-time. However, data based on these activities is often limited to use within the realm of the device manufacturers' ecosystems, and is thereby limited in scope.

Unfortunately, a majority of personal health-centric companies wishing to develop their own mobile device application, such as for a specific local gym or fitness organization to benefit their patrons, are often left with minimally-useful applications configured to only track a few basic metrics which are rarely customizable. If there were a platform by which such health-centric organizations could easily craft dynamic mobile device and web-based applications for their patrons which are configured to track and analyze health-based data with the assistance of A.I., organizations could more easily benefit their user's health. If such a platform employed easy-to-use modules which could be added to a created application without the need for programming prowess, more people would benefit from the advancements in health tracking and logging technologies on a personal level.

In short, the market is presently lacking in a useful application development platform oriented towards health-tracking metrics and the analysis thereof. No present mobile device or web-based application development platform focuses on personal health metrics while providing easy-to-use, pre-crafted modules suitable for use in a variety of applications. Thus, there is a need for a new application development platform and system which employs a variety of modules to facilitate the rapid creation of health-centric application for use by individual users without the need for advanced programming knowledge. Such a system is preferably equipped with an AI focused back-end, which ensures sound analysis of health-based input, and is configured to provide optimization suggestions to further an individual's health goals.

### SUMMARY OF THE PRESENT INVENTION

The present invention is an application development platform that facilitates the creation of custom-tailored applications via an integrated module-based system. The platform is configured to focus on the development of health, medical, sports, fitness, and well-being mobile device or web-based applications, however it may easily be used to create other module-based applications including markets, leadership management, and other goal-oriented, streamlining applications. The platform employs the use of Artificial Intelligence (AI) and an analytical system which recognizes, tracks, analyzes dependencies, correlations, patterns, and possible causes/consequences of differing evens and factors that may influence the overall health, performance, sport results, or well-being of the user, or that relate to the exchange of goods and services, or that pertain to leadership management and similar goal-oriented actions.

The modules of the platform are configured to provide suggestions to users of applications developed via the platform. Suggestions include tasks, exercises, diets, and medical advice pertaining to areas the system views as having the potential for improvement in order to obtain optimal results based on the personal data of the user. Additionally, the platform allows the creation of personalized health management tools (i.e. health-centric mobile device applications or web-apps) designed to reach health or sports goals based on the real-time data provided by the user.

The platform of the present invention provides a full cycle solution: data aggregation, goals/tasks creation/completion, and analysis. This solution engages all parties involved and leads to a result. However, depending on the industry, sector, and wanted results, modules included in the application created via the platform can differ, and their functions and numbers correspondingly differ as well. The modules are always combined in a way to create a full cycle solution for achieving something from the current state to a desired/planned outcome (i.e. to accomplish a goal). The whole process is tracked and transparent via the completed application as created and managed through the platform and system of the present invention.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1A depicts a chart of main modules and their relationships that base a core of the platform, showing relevant parts involved in the use and creation of applications via the platform of the present invention to be used by users.

FIG. 1B shows and lists the primary modules and how they interact on the platform, and inherent back-end of the platform and system of the present invention.

FIG. 2A exhibits a screenshot of an application crafted via the platform of the present invention, showing an exercise selection input screen.

FIG. 2B displays a screenshot of an application crafted via the platform of the present invention, showing a symptom selection input screen.

FIG. 3 depicts an example screenshot of a mobile device application crafted on the platform of the present invention which is geared towards stress reduction and the progression of goals involved thereof.

FIG. 4 depicts a flow chart detailing the progression of the Goals feature of the present invention as seen via the user experience through gamification.

FIG. 5 exhibits a diagram depicting the various system modules and sub-modules of the present invention, divided into related categories including client, client backend, core system, knowledge base, reporting, system testing, AI, service, and infrastructure.

FIG. 6 depicts a chart detailing use of the platform of the present invention for the development of a learning and/or leadership platform application.

FIG. 7 depicts a chart detailing the use of the platform of the present invention for the development of an anemia full cycle tracking and treatment solution application as an example health-oriented application.

FIG. 8 depicts a screenshot of an example embodiment of the marketplace portion of the system and platform of the present invention.

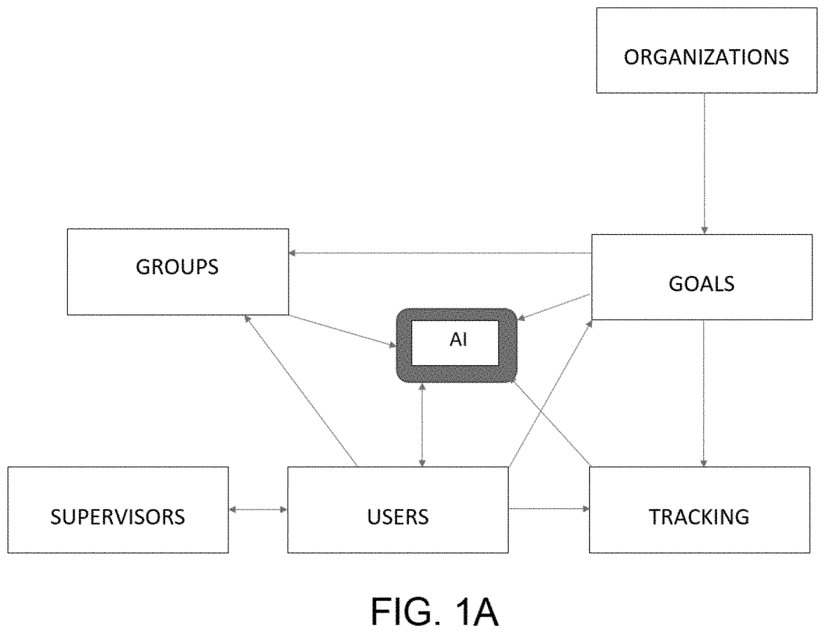

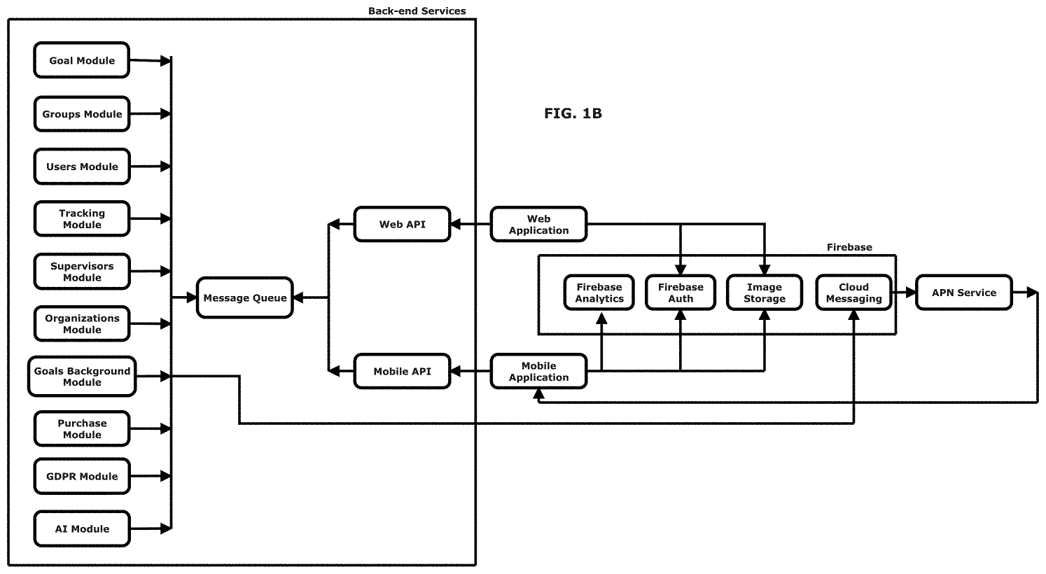

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s). The invention is defined by the claims appended hereto.

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to effect such feature, structure, or characteristic in connection with other embodiments whether or not explicitly described.

The present invention is a platform and system configured to facilitate the creation of module-based applications relating to personal health, including medical, sports, fitness, mental health, and well-being applications. Applications crafted via the platform of the present invention are preferably mobile device applications and/or web-based applications which are configured to be easy to use by any individual wishing to be more informed about his/her health and progression over time.

It is envisioned that the platform of the present invention is available for use via a conventional computer browser or may even employ a mobile browser. As such, the application development platform is preferably accessible online via a web portal or similar domain. The platform preferably employs custom APIs which are configured to interface with at least one server in communication with the platform. The at least one server is configured to remain in communication with the individual applications developed on the platform. At least one database is preferably also interfaced with the server to facilitate storage of tracked data and other user input provided in completed applications which were crafted via the system and platform of the present invention. Additionally, the AI and analytical system of the present invention is preferably executed on one or more of the servers, wherein user data is analyzed, and suggestions or ‘insights’ are conveyed to users via their respective installed applications.

It should be noted that any number of applications can be created with the platform of the present invention. All applications created via the platform preferably send and receive data to the servers, which, in turn, send and receive information from Web Panels and AI. In some embodiments of the platform, there might be several servers interfaced with the application(s) associated with the platform depending on the needs and application's specifications.

Similarly, in the preferred embodiment of the present invention, there are preferably several Web Panels available to users to facilitate the creation of their custom application (health-centric or otherwise). Web Panels are used to add, manage, provide, review, adjust, supervise, and control data within applications and to perform other various functions. There might be any number of AI modules and sub-modules depending on the needs of the application in development. AI and analytical system modules are configured to receive, manage, analyze the data from users. The data provided through research and other means provides insights, suggestions, dependencies, correlations, patterns, possible causes and consequences of different events and factors that might have influenced health, performance, sport results or well-being of users. As such, the applications developed via the system and platform of the present invention are configured to predict possible health issues and sports injuries before they actually occur.

There are preferably 33 modules available for a developer to employ in the creation of a health-centric mobile device application (or web-based application) via the present invention. It should be understood that each module may be used independently, but each is configured to work together with all other modules. The modules may all be built into a single application together, separately, or in any combination.

Modules available for integration into applications developed via the platform and system of the present invention preferably include, but are not limited to, the following:
Task tracking modules, including those oriented to the tracking of physical exercises, nutrition, medication use, supplements/vitamins intake, mindset, and symptoms;
Non-tracking tasks, including general well-being, optimism/pessimism levels, journaling, etc.
Input modules, configured to interface with known health tracking systems such as FitBit™, Apple™ Watch, internet-connected scales, and other similar personal fitness trackers.
Medical input modules configured to provide quantitative data pertaining to heart rate and/or blood conditions including diabetes.
Medical Organization input module, configured to enable medical institutions such as doctors offices and hospitals to provide medical data relating to the user to the application.
Manual input modules, configured to enable the user to manually write into the application based on activities/exercises performed, diet (i.e. hunger on a scale of 1-10), and other personal metrics that could be quantitated.

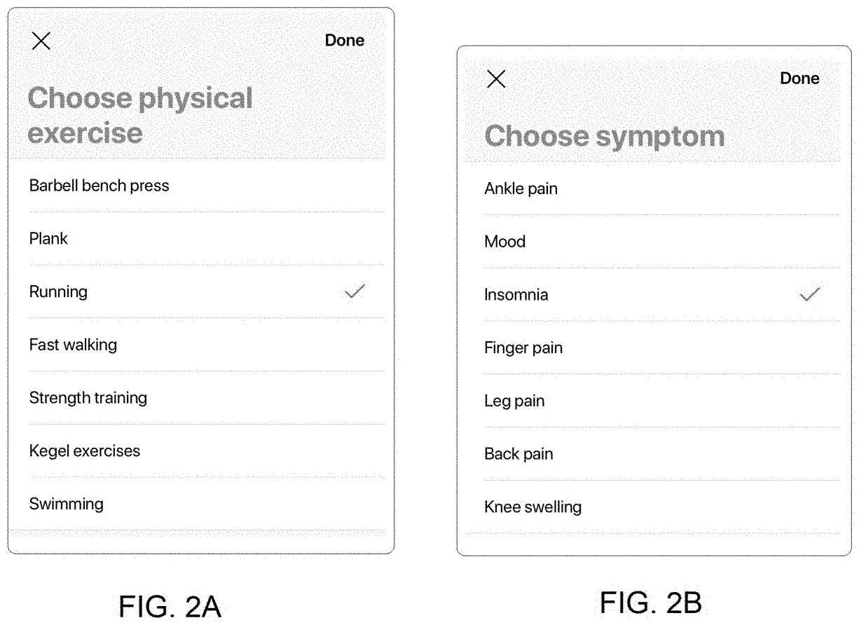

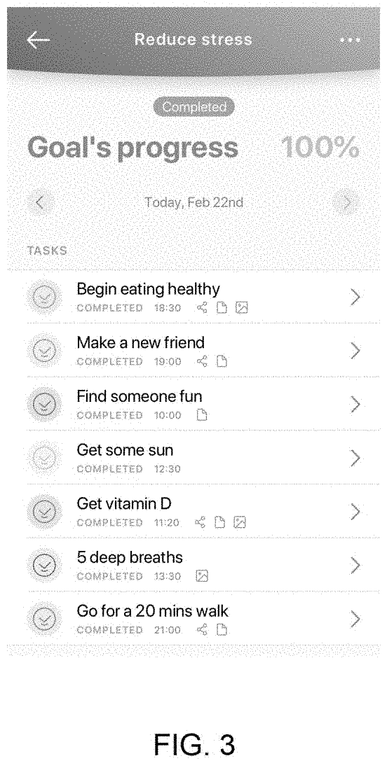

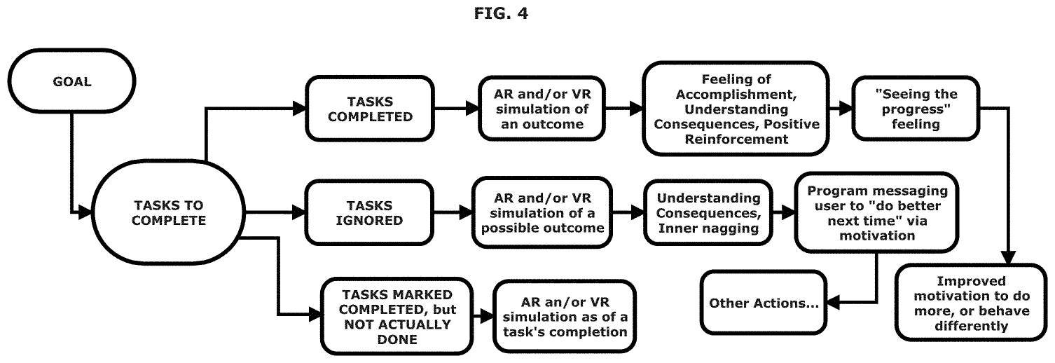

An example usage scenario of an application professionally created via the platform of the present invention may include:
1. A user employing a health-oriented application configured to track the user.
2. The user manually inputting health supplement intake numerically into an input field of the application.
3. The user posing symptoms to the application. FIG. 2B exhibits an example input for symptoms of the user.
4. The application storing the input to a database.
5. The application conferring with the input of the database and employing the server to research information for a suggestion to be made to the user based on the occurrence and frequency of the symptoms provided. For example, the system would make a suggestion of increasing or decreasing the health supplement, or may note to the user that certain probiotic supplements can increase constipation or can cause diarrhea, stating the commonality of the symptom experienced and logged.
6. The user adjusting his/her intake of the health supplement based on the suggestion(s) from the application.

An example of an application crafted via the platform of the present invention may be seen via a health management tool, referenced as HealthCraft by example. Such a tool preferably provides users personalized analytics based on an assortment of health metrics which can help the users to reach any sports/health goals. Such an application may be adjusted in order to fit the needs of the user such that any coach, doctor, or health expert can create and assign goals to users via an associated web panel. Then, the user receives his or her goals/tasks via the mobile device application, and is encouraged to complete them through the application. Completion of goals may be reviewed in real-time by the application administrator (preferably the coach, doctor, or health expert). This example application, as well as most others, allows both the administrator and the end user to track symptoms, habits, physical exercise, nutrition, medications taken, and supplements taken in one place—the application. Based on this input, the user can then receive personalized analytics based on the user's input data which can be used to make optimal health decisions in order to excel performance and health.

It should be understood that the more data that the user provides to his/her application (over a long period of time) and the more accustomed/trained the AI is to the quirks and needs of the user, the better insights and suggestions are that are provided to the user in order to act towards the benefit of the user's health. Photographic data, such as pictures of the end user, may also be used by the AI of the applications. For example, based on pictures and medical data of the user, the system may be configured to detect the presence of an oncoming migraine based on a facial expression of a recently provided photo.

Additionally, it should be understood that the platform of the present invention provides for additional modules to be added for use by users at any time in the future based on the development of health tracking technologies. Similarly, it should be noted that embodiments of the present invention are configured for the development of leadership management, marketplace management, and other goal-oriented, task-specific development tools in application format.

As such, it should be noted that the present invention allows third parties (a parent, a nurse, a coach, etc.) to track a health data and use it to diagnose and track treatment success. The present invention also includes a new system dialect FHIR (Fast Healthcare Interoperability Resources) module, which is needed to show the system's practical implementation in making healthcare accessible to all people be it a child, an adult, or a senior living in a city or rural area.

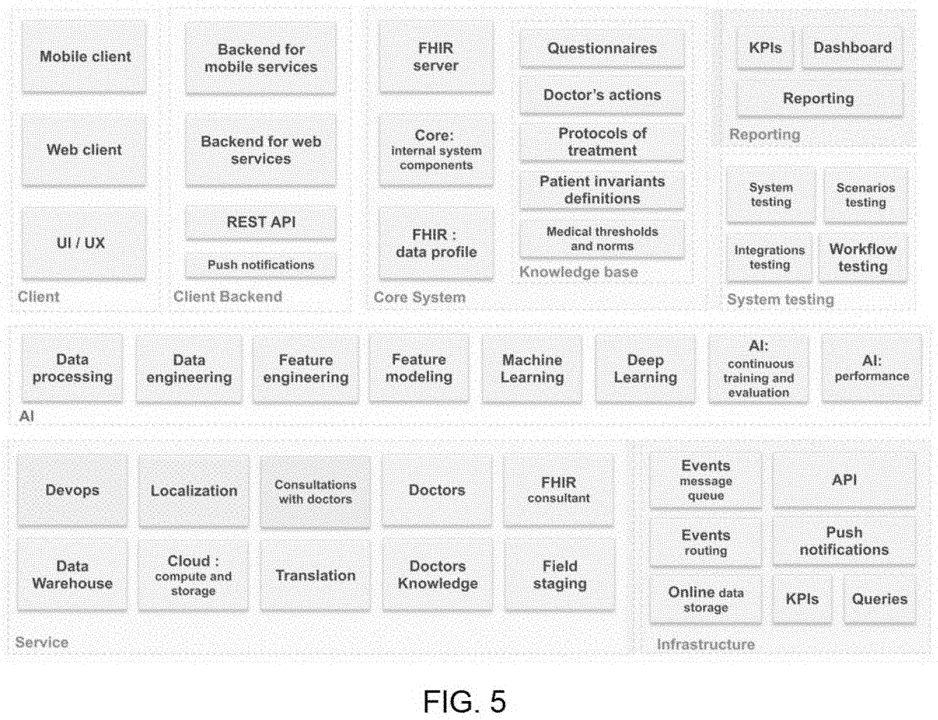

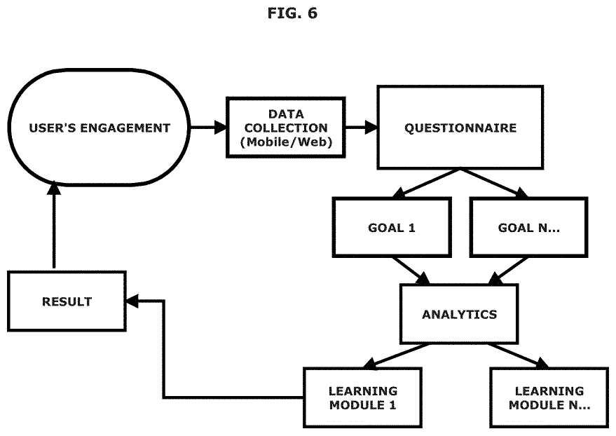

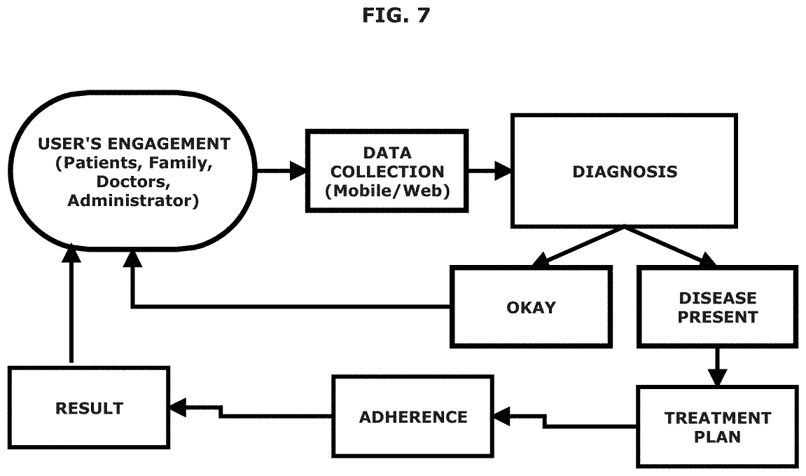

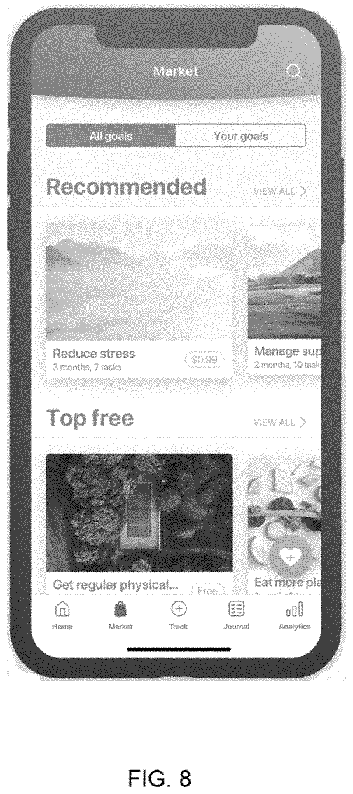

Various modules available via the application development system and platform of the present invention can be seen in FIG. 5. Modules are depicted within dotted blocks, with the name of each module. Submodules are given as a group of blocks within each module. Details pertaining to the modules and sub-modules of the platform include:

Client service applications: clients interfaces.

The purposes of the client service application modules are:
To collect the data from patients
To present the working materials for doctors
To present the doctor decisions or other objective system info for patients
To present the KPIs and reports to administrator.

### Submodules:

mobile for Patients
Web for Doctors and Administrator
UI/UX for all client interfaces
   Clients service backend (for both the Web and Mobile clients platforms). The purposes are to define and serve system scenarios for client services frontend applications.

### Submodules:

Web applications backend
Mobile applications backend
APIs (REST)
Push notifications
   Core System: defines data profiles and components that use them, including internal components. Includes medical knowledge base created and managed in cooperation with the Doctors and Administrators.

### Submodules:

FHIR:
   Schema profiles
   FHIR messaging profiles
   FHIR server integration
      Knowledge base
      Submodules: medical knowledge of the system:
Questionnaires
Doctor actions
Medical protocol of treatment
Patients invariants definitions (age, sex, height, weight, etc.)
Medical thresholds and norms
Infoboard
Symptoms
Risk factors
Dietary recommendations
   Reporting: System performance dashboard based on KPIs+infoboard for patients.

### Submodules:

KPIs definitions and numbers
Reporting
Dashboard
Infoboard definitions interface
   AI: group that develops the RAH automatic intelligence, the modeling infrastructure, objective research, experiments and modeling itself.

### Submodules:

Data integration
Data processing
Data engineering
Feature engineering
Feature modeling
Machine Learning
Deep Learning
Modeling ensemble continuous train and evaluation
Modeling performance evaluation
Modeling performance reporting
   Testing: continuous integration system testing, sub-moduled by:
System testing
Integrations testing
Scenarios testing
Workflow testing
A part of field testing

Another example usage of the application development platform and system of the present invention includes an Anemia full cycle tracking and treatment solution application as seen in FIG. 7. Details pertaining to such an example application are as follows:

The anemia solution example, as shown in-short in FIG. 7, further expands on the platform's ability to analyze, compare and find correlations with a new advanced function to non-invasively diagnose as well as track cause & effect factors simultaneously. The AI performs several steps using its integrated submodules. Specifically, it analyzes the main area detection (eye, nail), establishes a detection of “diagnostic area”. If it is not present, the system requests for photos to be retaken. Then, if area is found, it runs appropriate detection model and gets both diagnosis and a level of confidence. If level of confidence is high, the system reports a result to the user. If level of confidence is low, there is a recommendation to turn to a human expert. The detection models themselves are preferably constitutional neural networks, which cannot give too much explanation. However, heat-maps will be provided: areas of photo which contributed most to the diagnosis.

To further elaborate on the anemia solution example as created via the platform of the present invention, the objective is to track, diagnose, facilitate treatment of anemia in patients. To accomplish these goals, the system must be equipped with:
Instrument for data collection (from patients)
Instrument for data analysis (screening)
Instrument for data labeling (diagnosis and decisions)
Instrument for screening and recommendation help delivery (to the patients)
Instrument for measurement of the performance of the solution.
Instrument of expert system learning.
Instrument for performance reporting.
Instrument to maintain all features and their usage together for system to provide the services.

As such, the System consists of the set of Modules that are dedicated to interact according to scenarios defined by the System state machine to achieve the target objectives:
To collect the data from remote Patients.
To process and forward the data:
to Expert System for making the decision on this data
To the warehouse for the History (the dataset)
To propagate the Expert decision
to the issued side in the form of Response
To the warehouse for the History and labels (the dataset)
To collect the statistics that evolve as a result of the System performance.
To report statistics.
To learn AI to be an Expert in the Anemia objective and mediate in a role of a Doctor.

Therefore, the system should provide the fixed set of KPIs, in raw form and in the form of reports. Data Science works are intended to grow the modeling ensemble of the AI to provide the remote anemia help AI-driven solution. This concept is represented by scopes:
Integration on data nature given with the objective-related data samples
Labeled photos of patient's nails
Patients invariants
Patients lab results
Consultation with medical experts on objective and the data relation to the objective.
Building the data acquisition pipeline for model's train and test phases.
Feature engineering: build data processing pipeline for continuous model training and test cycles.
Build the modeling framework for the objective. Modeling framework approaches:
Machine Learning techniques
Deep Learning (Deep AI)
Build the pipeline for modeling ensemble performance evaluation and reporting.

Additionally, the system considers the locality where it will be used. This includes language, human perception specifics, and cultural/racial features. Via the modules as depicted in FIG. 5, the system processes and records all the data which is streamed through it in a standardized and flexible format (FHIR). This provides the integrability with other medical data, medical structures and applications. Various user data is collected via the application crafted and managed by the platform and system of the present invention, including contact information, children information, medications, and anemia related data (symptoms, risk, factors, prevention actions, dietary actions, etc.). An information panel is preferably present in the crafted client application to indicate to user's general information pertaining to Anemia (risk factors, prevention notes, and dietary recommendations). Similarly, the crafted application is preferably configured to display any and all lab results pertaining to the tests administered. A doctor's dashboard, viewable only by qualified personnel, is preferably available via a separate application or web-based portal, which aggregates and displays pertinent information relating to the patients at hand relating to the issue(s) tracked.

A primary component of the present invention which helps to differentiate it from other application development platforms present on the market is its goal-oriented approach to completing and tracking tasks in a transparent fashion. As such, goals-based submodules pertaining to the creation and tracking of goals are inexorably linked with a variety of other modules. Namely, the goals module itself is a module that allows one to create goals (an idea of a desired outcome consisting of tasks). Users of applications equipped with the goals module (such as marketplace applications) could get a goal (a pre-set goal/task/job) from a marker, or create private custom goals as needed for leadership/organization. When a user begins work on a pre-set goal, tasks that pertain to the goal are provided within the goal for the user to see. Users cannot make any changes (add new tasks, change frequency of tasks, etc.) to a pre-set goal obtained from the marketplace.

Preferably, each goal as outlined in the application development platform of the present invention has the following attributes:
Unique ID
Rating
Tags
Title
Image
Description
List of Tasks
Sharing parameter {Private, Public}
Publish Status {Waiting for Approve, Approved, Rejected}
Price
Duration (in days)
Progress
Status {Available, Active, Suspended, Completed}
   Correspondingly, each task of each goal preferably has the following attributes:
Unique ID
Title
Description
Tracking entity
Schedule
Related Goal
Status {Available, Active, Ignored, Completed}
Completion information (text, image)
   Logic of Goal and Task progress calculation:
Each task will be assigned a specified number of points. When a goal is set with certain tasks and #of them to be completed, there will be a total number of points to be gained for all the tasks within the goal. Based on the % of total points gained so far, will be estimated the % of the goal achieved. Example: my goal has 10 tasks. 5 of them the user has to complete 3 times each (15). So, for example, in total, a user will have 20 tasks. When the user completes a task, his/her goal gets 5% completed.
If a task is ignored, user does not get any number of points, and progress of Goal is not changed.
   Logic of Goal's versions:
Each Goal could have several versions;
New version of the Goal should be created automatically, when a Goal Creator publish it.
For all users should be shown and used latest version of the Goal with Publish Status—Approved.
User could start work on a Goal from market, if the Goal is Approved and Sharing Parameter is not Private
   Logic of Goal publishing:
If a Goal creator changes Sharing parameter from Public to Private, for users, who have already worked on the Goal, Goal should be kept and available.
If a Goal has a sharing parameter Public, Goal should be approved by the admin.
If in new Goal's version some tasks are deleted, user should not be able to see deleted tasks and their completion history. However, if deleted task was a tracking task, all tracking should be available for user in the tracking section.
If the admin changes Goal's Publish Status to Rejected, he should specify a reason, why it's rejected, and Goal Creator should be notified about it.
If the admin changes Goal's Publish Status to Approved, Goal is available for other users in the market.

### Logic of Ratings:

When a user has completed a Goal, he should be able to rate a Goal
After first completion of the Goal, user could change rating of a Goal anytime.

The status of each goal is relevant to its current level of completion, and as to whether the tasks comprising each goal may be completed in the time required. As such, each task of each goal is assigned a status.

Available changes of statuses for Goals:

| From | To | By whom | When |
|---|---|---|---|
| Available | Active | Service | Goal is started for User |
| Active | Suspended | User | User decides to suspend a Goal |
| Active | Suspended | Service | Automatic change of Goal's |
| | | | status by the service |
| Active | Completed | Service | Automatic change of Goal's |
| | | | status by the service |
| Suspended | Active | User | User decides to re-start a |
| | | | Goal (Goal should be started |
| | | | from the begging). |
| Completed | Active | User | User decides to repeat a Goal. |

Available changing statuses for Tasks:

| From | To | By whom | When |
|---|---|---|---|
| Available | Active | Service | Goal is started for User |
| Active | Ignored | User | User decides to ignore a task |
| Active | Ignored | Service | Automatic change of Task's |
| | | | status by the service |
| Active | Completed | User | User has completed a task |
| Completed | Active | User | User decided to repeat a task |

It should be understood that such market-oriented goal-based applications developed via the platform and system of the present invention similarly employ AI for the tracking and motivation of the completion of said goals. Similarly, the system is preferably configured to automatically change the status of a goal/task/job, as well as confirm any and all manual status changes. For example, after each change of task's status to “Ignored,” there should be a check to determine whether the goal could be completed in the future (% of Ignored tasks from all possible tasks<=20). If not, the system will issue a notification to user and goal should be marked as “Suspended.” Likewise, after each change of a task's status to “Completed” or “Ignored” there should be a check to determine whether user has tasks with a status of “Available” in this goal. If the user does not have any tasks in the goal with a status of “Available,” the status of a goal should be changed to “Suspended” (% of Ignored tasks from all possible tasks<=20) or “Completed” (% of Completed tasks from all possible tasks=>80). When a goal duration is over, the status of a goal must be changed to “Completed” status (% of Completed tasks from all possible tasks=>80) or to “Suspended” (any other conditions).

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A modular application development platform and system facilitating the custom creation of at least one device application comprising:
a first device gathering data pertaining to an individual;
a second device storing the gathered data, relating the data to an account of the individual;
wherein the data pertains to health of the individual;
artificial intelligence categorizing the data as it relates to pertinent health usage applications;
the artificial intelligence analyzing the data; and
the artificial intelligence positing suggestions to the individual to better the health of the individual.

2. The system of claim 1, wherein the at least one device application is composed of interdependent modules; and
wherein the interdependent modules facilitate adaptability of the platform to function as discrete instances of at least one device application.
